(19) United States
(12) Patent Application Publication
(10) Pub. No.: US 2025/0388963 A1
(43) Pub. Date: Dec. 25, 2025

(54) PRIMER DESIGN FOR CELL-FREE DNA PRODUCTION

(71) Applicant: ModernaTX, Inc., Cambridge, MA (US)

(72) Inventors: Kevin Smith, Cambridge, MA (US); David Schulz, Cambridge, MA (US)

(73) Assignee: ModernaTX, Inc., Cambridge, MA (US)

(21) Appl. No.: 18/881,066

(22) PCT Filed: May 5, 2023
(86) PCT No.: PCT/US2023/066633

§ 371 Date: Jan. 3, 2025

Related U.S. Application Data
(60) Provisional application No. 63/358,636, filed on Jul. 6, 2022.

(51) Int. Cl. C12Q 1/6876; C12Q 1/6806

(52) U.S. Cl. CPC C12Q 1/6876; C12Q 1/6806

(57) ABSTRACT

The present disclosure generally relates to the use of linear nucleic acid primers for the amplification of a target nucleic acid sequence, for example, in a cell-free environment. In some embodiments, compositions of the linear nucleic acid primers are provided. For example, in some embodiments, the linear nucleic acid primers comprise a guanosine or a cytidine at 3′ terminal end. In some embodiments, the linear nucleic acid primers have been optimized to prevent primer-homodimer and/or hairpin formation and to exclude cumbersome codon sequences. In some embodiments, methods are provided for the amplification of a DNA template fragment using the linear nucleic acid primers. Thus, in some cases, the use of the nucleic acid primers, as described herein, may allow for the reduction in amplification of non-specific hybridization events while allowing for the amplification of the target nucleic acid sequence.

### RELATED APPLICATIONS

This application claims priority under 35 U.S.C. § 119 (e) to U.S. Provisional Application, U.S. Ser. No. 63/358,636, filed Jul. 6, 2022, which is incorporated herein by reference.

### GOVERNMENT LICENSE RIGHTS

This invention was made with Government support under Agreement No. HR0011-20-9-0118, awarded by DARPA. The Government has certain rights in the invention.

### REFERENCE TO AN ELECTRONIC SEQUENCE LISTING

The contents of the electronic Sequence Listing (M137870235WO00-SEQ-HCL.xml; Size: 10,101 bytes; and Date of Creation: May 3, 2023) are herein incorporated by reference in their entirety.

### FIELD

The present disclosure generally relates to methods and compositions useful in cell free production of deoxyribonucleic acid.

### BACKGROUND

Messenger RNA (mRNA) is an emerging alternative to conventional small molecule and protein therapeutics due to the potency and programmability of mRNA. mRNA encoding a desired therapeutic or prophylactic protein can be administered to a subject for in vivo expression of the protein, for use in a method such as vaccination or replacement of a protein encoded by a mutated gene. In vitro transcription (IVT) of a DNA template using an RNA polymerase is a useful method of producing mRNAs. The process uses a DNA template to achieve quality commercial scale mRNA.

One method for producing DNA templates for IVT involves gene synthesis, a process of assembling shorter nucleic acid sequences (i.e., fragments of the DNA template) into the desired DNA template structure. This requires amplification of the shorter nucleic acid sequences (i.e., DNA template fragments) using techniques such as polymerase chain reaction (PCR), in which a set of primers bind to the DNA template fragment via Watson-Crick base pairing and direct elongation toward opposite ends of the target sequence being amplified. Despite the development of a set of universal guidelines to enhance hybridization efficiency, primer design remains an inexact science and often requires multiple iterations before an acceptable degree of amplification and purity of the DNA template fragment is achieved. Thus, improvements are needed.

### SUMMARY

The present disclosure relates to methods and compositions for cell free production of deoxyribonucleic acid. The subject matter of the present disclosure involves, in some cases, interrelated products, alternative solutions to a particular problem, and/or a plurality of different uses of one or more systems and/or articles.

In some embodiments, the present disclosure relates to a nucleic acid primer, comprising a nucleic acid having a 5′ terminal end and a 3′ terminal end and a polynucleotide sequence of 20 to 40 nucleotides, wherein the polynucleotide sequence comprises a guanosine or a cytidine at the 3′ terminal end.

In some embodiments, the nucleic acid primer comprising a polynucleotide sequence has between about 32 and 38 nucleotides.

In some embodiments, a nucleic acid primer comprises a polynucleotide sequence, wherein the polynucleotide sequence comprises a GC content of about 50%.

In some embodiments, a nucleic acid primer comprises a polynucleotide sequence, wherein the polynucleotide sequence has a low primer-homodimer complex forming propensity.

In some embodiments, the primer-homodimer complex forming propensity comprises a delta G of greater than or equal to −3.0 kcal/mol.

In some embodiments, a nucleic acid primer comprises a polynucleotide sequence, wherein the polynucleotide sequence has a low hairpin structure forming propensity.

In some embodiments, the hairpin structure forming propensity comprises a delta G of greater than or equal to −2.5 kcal/mol.

In some embodiments, a nucleic acid primer comprises a polynucleotide sequence, wherein the polynucleotide sequence is

(SEQ ID NO: 1)
TCTGGACGGACGCTTCGGACGATGGAACAATTCAGTG.

In some embodiments, a nucleic acid primer comprises a polynucleotide sequence, wherein the polynucleotide sequence is

(SEQ ID NO: 2)
AGCGGTGTATACGGTGTAAACACTTCGACGCTTTCCGG.

In some embodiments, a nucleic acid primer comprises a polynucleotide sequence, wherein the polynucleotide sequence is

(SEQ ID NO: 3)
AGTGCGACATGGTACTTTTCTGTGATCGCTCGCCTCG.

In some embodiments, a nucleic acid primer comprises a polynucleotide sequence, wherein the polynucleotide sequence is

(SEQ ID NO: 4)
CCGCAAGCCGCTCCTTGAATCTACGGAGAGACTCAC.

In some embodiments, a nucleic acid primer comprises a polynucleotide sequence, wherein the polynucleotide sequence is AATCGTCGCCGTCCTCACAAAAACAACCGCCG (SEQ ID NO: 5).

In some embodiments, a nucleic acid primer comprises a polynucleotide sequence, wherein the polynucleotide sequence is

(SEQ ID NO: 6)
CGCCGAGGCTAAATCGCAATCTACCTGACGTTCCTGTG.

In some embodiments, a nucleic acid primer comprises a polynucleotide sequence, wherein the polynucleotide sequence is

(SEQ ID NO: 7)
ATCGACTTGCCTGCTGTCATTACTTCACGCTCACTCCG.

In some embodiments, a nucleic acid primer comprises a polynucleotide sequence, wherein the polynucleotide sequence is

(SEQ ID NO: 8)
CGTACAGTGACCTATCGCCAGAATCTCACGCCAACAGC.

In some embodiments, a nucleic acid primer comprises a polynucleotide sequence, wherein the polynucleotide sequence is

(SEQ ID NO: 9)
CGGAGAAGCCAATCAGGTCCTTGATTCTCTACCAGCGC.

In some embodiments, a nucleic acid primer comprises a polynucleotide sequence, wherein the polynucleotide sequence is

(SEQ ID NO: 10)
TTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTT
TTGCCGCCCACTCAGACTTTATTCAAAGACCACGGGG.

Aspects of the current disclosure relate to methods of preparing a DNA template fragment, comprising:
(i) hybridizing a plurality of DNA templates with a first set of primers to produce a first set of DNA template-first primer duplexes,
   wherein the first set of primers comprises two or more primers, and wherein each of the primers in the first set of primers comprises a polynucleotide sequence having a guanosine or a cytidine at a 3′ terminal end, and at least one nucleotide that is unique to that primer and different from each of the other primers in the first set of primers;
(ii) extending the first set of DNA template-first primer duplexes in a 5′ to 3′ direction and a 3′ to 5′ direction to produce a first extension product;
(iii) hybridizing single strands of the first extension product to a second set of primers, to produce a second set of DNA template-second primer duplexes, wherein the second set of primers comprises two or more primers, and wherein each of the primers in the second set of primers comprises a polynucleotide sequence having a guanosine or a cytidine at a 3′ terminal end and at least one nucleotide that is unique to that primer and different from each of the other primers in the second set of primers
(iv) extending the second set of DNA template-second primer duplexes in both the 5′ to 3 direction and the 3′ to 5′ direction to produce a second extension product; and
(v) allowing the first extension products to hybridize with the second extension products in both the 5′ to 3′ direction and the 3′ to 5′ direction to produce the DNA template fragments.

In another aspect, the present disclosure encompasses methods of making one or more of the embodiments described herein, for example, cell free production of deoxyribonucleic acids. In still another aspect, the present disclosure encompasses methods of using one or more of the embodiments described herein, for example, cell free production of deoxyribonucleic acids.

Other advantages and novel features of the present disclosure will become apparent from the following detailed description of various non-limiting embodiments of the disclosure when considered in conjunction with the accompanying figures.

### DETAILED DESCRIPTION

Provided are compositions of linear nucleic acid primers and methods of amplifying target nucleic acid sequences. Target nucleic acid sequences are typically amplified in vitro using polymerase-chain reactions (PCR), in which a set of primers (e.g., a forward primer and/or reverse primer) bind to the target nucleic acid sequence (e.g., DNA) via Watson-Crick base pairing and direct elongation toward opposite ends of the target sequence being amplified. The design of the primer sequences (e.g., forward primers and/or reverse primers) is important for achieving successful DNA amplification. For example, poorly designed primers may result in nonspecific DNA amplification products, truncated sequences, primer-homodimer formation, primer hairpin formation, etc.

To overcome these issues, the field has adopted a general set of guidelines to help guide successful primer design. However, despite these guidelines, primer design remains an inexact science and often requires multiple iterations before an acceptable degree of amplification and purity of the amplicon (i.e., the target nucleic acid target) is achieved.

### Primer Design

Provided are a set of rules for the selection of a set of nucleic acid primers that may be used to amplify any target nucleic acid sequence (e.g., a DNA template encoding an mRNA vaccine and/or therapeutic), which solve the aforementioned problems. In some embodiments, the rules comprise creating a library of randomly generated primer sequences. The length of the nucleic acid primer sequence may vary. For example, in some embodiments, the nucleic acid primer may have a length of between 10 nucleotides to about 50 nucleotides, of between 15 nucleotides to about 45 nucleotides, of between 20 nucleotides to about 40 nucleotides, of between 25 nucleotides to about 35 nucleotides, of between 32 nucleotides to about 38 nucleotides, etc. In some embodiments, the nucleic acid primer may have a length of at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, etc. In some embodiments, the length of the nucleic acid primer sequence is between 20 and 40 nucleotides; in other embodiments, the length of the nucleic acid primer sequence is between 32 and 38 nucleotides.

In some embodiments, a randomly generated library of nucleic acid primer sequences comprises greater than or equal to 1×10\hat{ }6 primer sequences, greater than or equal to 2×10\hat{ }6 primer sequences, greater than or equal to 3×10\hat{ }6 primer sequences, greater than or equal to 4×10\hat{ }6 primer sequences, greater than or equal to 5×10\hat{ }6 primer sequences, etc. In other embodiments, the library comprises less than or equal to 5×10\hat{ }6 primer sequence, less than or equal to 4×10\hat{ }6 primer sequences, less than or equal to 3×10\hat{ }6 primers sequences, less than or equal to 2×10\hat{ }6 primer sequences, etc.

In some embodiments, the rules comprise filtering out primers with problematic sequences from a library of randomly generated primers. For example, primers with a high GC content may favor primer-homodimer formation, therefore, in some embodiments, the GC content of the nucleic acid primer sequence is about 30%, about 40%, about 50%, about 60%, of the total number of nucleotides in the nucleic acid primer. In some embodiments, the GC content of the linear nucleic acid primer sequence is 50% of the total number of nucleotides in the primer sequence. In some cases, the linear nucleic acid primer sequence comprises either a G or C nucleotide at a 3′ end.

In some cases, primers comprising GC-rich and/or AT-rich domains may be filtered from the library as they may adopt secondary structures in solution, thus decreasing hybridization efficiency. In other embodiments, primers comprising dinucleotide repeats (e.g., ATATAT) and/or runs of 4 or more of a particular bases (e.g., AAAA, TTTT, CCCC, or GGGG) may be excluded from the library. In some embodiments, primers comprising more than 3 bases that complement within the primer (e.g., intra-primer homology) may also be screened out to reduce the likelihood of hairpin formation (e.g., self-dimer); forward and reverse primers having complementary sequences may be removed from the library as they favor primer-homodimer formation over annealing to the desired nucleic acid target sequence. In some embodiments, triplet domains that are troublesome to incorporate using synthetic techniques may also be excluded to increase yields when produced, for example, using a nucleic acid synthesizer.

In some embodiments, the rules comprise selecting primers with a desired melting temperature (Tm). Any method known to those of ordinary skill in the art may be used to calculate the Tm. For example, in some embodiments, the method uses a formula where the Tm depends only on the relative content of cytosine and guanine in the primer sequence as described in Marmur et al (J. Mol. Biol. 1962; 5109-118). In other cases, the Tm may be calculated using an improved formula that contains a correction factor that accounts for the contributions of various experimental parameters (e.g., salt concentration) as described in Wetmur (Crit. Rev. Biochem. Mol. Biol. 1991; 26227-259). In other embodiments, a Nearest Neighbor (NN) model may be used, wherein the NN model accounts for the relative amount of cytosine and guanine in the sequence as well as the sequential arrangement of different nucleotides in the primer sequences, which plays a key role in the thermodynamics of hybridization. Several tables with DNA/DNA thermodynamic parameters for use in the NN model are described, for example, in Allawi et al (Biochemistry. 1997 Aug. 26; 36 (34): 10581-94), Gotoh et al (Biopolymers. 1981; 201033-1042), and Sugimoto et al (Nucleic Acids Res. 1996; 244501-4505), among others.

In some embodiments, the rules comprise selecting primers with a melting temperature (Tm) of greater than or equal to 60° C., greater than or equal to 64° C., greater than or equal to 68° C., greater than or equal to 70° C., greater than or equal to 75° C., greater than or equal to 78° C., etc. In some embodiments, an algorithm comprises selecting primers with a melting temperature of less than or equal to 78° C., of less than or equal to 75° C., of less than or equal to 70° C., of less than or equal to 68° C., or of less than or equal to 64° C., etc.

In some embodiments, the rules comprise using a melting temperature (Tm) to reduce the number of primers in the library from between 1×10\hat{ }6 to 5×10\hat{ }6 nucleic acid primers to greater than or equal to 500 primers, greater than or equal to 750 primers, greater than or equal to 1000 primers, greater than or equal to 1250 primers, greater than or equal to 1500 primers, greater than or equal to 1750 primers, greater than or equal to 2000 primers, greater than or equal to 2500 primers, greater than or equal to 3000 primers, etc. In some embodiments, the rules comprise using the Tm to down select the library of primers from between 1×10\hat{ }6 to 5×10\hat{ }6 nucleic acid primers to less than or equal to 3000 primers, less than or equal to 2500 primers, less than or equal to 2000 primers, less than or equal to 1750 primers, less than or equal to 1500 primers, less than or equal to 1250 primers, less than or equal to 1000 primers, less than or equal to 750 primers, less than or equal to 500 primers, etc.

In other embodiments, the rules comprise determining a change in a Gibbs free energy (delta G) of a primer sequence selected from a primer library at a desired melting temperature (Tm). Methods of calculating the Gibbs free energy are generally known by those of ordinary skill in the art and may be used to predict and filter out sequences prone to primer-homodimer and/or hairpin formation. Briefly, delta G represents the quantity of energy needed to break any secondary structures adopted by the primer (e.g., primer-homodimer and/or hairpin). For example, a lower delta G value (i.e., more negative values) suggests the presence of secondary structure (e.g., primer-homodimer and/or hairpin formation) as more energy is needed to separate the DNA strands, relative to a primer sequence that does not form such secondary structure.

In some embodiments, a delta G for a nucleic acid primer homodimer, at a temperature between 60° C. and 78° C., is greater than or equal to −9 kcal/mol, greater than or equal to −5 kcal/mol, greater than or equal to −4 kcal/mol, greater than or equal to −3.5 kcal/mol, greater than or equal to −3 kcal/mol, greater than or equal to −2.5 kcal/mol, greater than or equal to −2 kcal/mol, greater than or equal to −1.5 kcal/mol, greater than or equal to −1 kcal/mol, etc. In some embodiments, the delta G for a nucleic acid primer homodimer is less than or equal to −1 kcal/mol, less than or equal to −1.5 kcal/mol, less than or equal to −2 kcal/mol, less than or equal to −2.5 kcal/mol, less than or equal to −3 kcal/mol, less than or equal to −3.5 kcal/mol, less than or equal to −4 kcal/mol, or less than or equal to −5 kcal/mol, etc.

In some embodiments, a delta G for a nucleic acid primer hairpin, at a temperature between 60° C. and 78° C., is greater than or equal to −9 kcal/mol, greater than or equal to −5 kcal/mol, greater than or equal to −4 kcal/mol, greater than or equal to −3.5 kcal/mol, greater than or equal to −3 kcal/mol, greater than or equal to −2.5 kcal/mol, greater than or equal to −2 kcal/mol, greater than or equal to −1.5 kcal/mol, greater than or equal to −1 kcal/mol, etc. In some embodiments, the delta G for a nucleic acid primer hairpin is less than or equal to −1 kcal/mol, less than or equal to −1.5 kcal/mol, less than or equal to −2 kcal/mol, less than or equal to −2.5 kcal/mol, less than or equal to −3 kcal/mol, less than or equal to −3.5 kcal/mol, less than or equal to −4 kcal/mol, or less than or equal to −5 kcal/mol, etc.

In some embodiments, the rules comprise validating the nucleic acid primers, for example, using quantitative polymerase chain reaction (qPCR). Any method known to those of ordinary skill in the art may be used to validate the nucleic acid primers, such as, for example, qPCR. In some embodiments, the validation comprises preparing one or more test samples comprising a nucleic acid target sequence of interest (e.g., a DNA template) and the optimized primer set (e.g., optimized forward and reverse primer set). In some cases, the concentration of the target nucleic acid in the test sample may be between 10 ng/uL and 50 ng/ml. In some embodiments, the concentration of the primer set (i.e., the forward and/or reverse primer) in the test sample is between 100 nM and 500 nM. In some embodiments, the primer concentration in the test sample is greater than or equal to 100 nM, greater than or equal to 200 nM, greater than or equal to 300 nM, greater than or equal to 400 nM, greater than or equal to 500 nM, etc. In other embodiments, the primer concentration in the test sample is less than or equal to 500 nM, less than or equal to 400 nM, less than or equal to 300 nM, less than or equal to 200 nM, less than or equal to 100 nM, etc.

In some embodiments, validation of a pair of nucleic acid primers comprises performing a thermal gradient analysis using, for example, qPCR. As will be understood by those of ordinary skill, the thermal gradient analysis may be used to identify the optimal annealing temperature (typically identified as the temperature at which the amplification curves overlap). In some cases, the temperature of the thermal gradient analysis may be greater than or equal to 50° C., greater than or equal to 55° C., greater than or equal to 60° C., greater than or equal to 65° C., greater than or equal to 70° C., greater than or equal 75° C., greater than or equal to 80° C., etc. In other embodiments, the temperature of the thermal gradient analysis is less than or equal to 80° C., less than or equal to 75° C., less than or equal to 70° C., less than or equal to 65° C., less than or equal to 60° C., less than or equal to 55° C., less than or equal to 50° C., etc. In some embodiments, the amplicon is further analyzed to ensure the correct product is being amplified, for example, by performing a melt curve analysis (e.g., to ensure a single amplicon is present) and/or gel electrophoresis (e.g., to ensure correct molecular weight of amplicon).

In some embodiments, validation of a pair of nucleic acid primers comprises generating a standard curve using, for example, qPCR. In some cases, samples for the standard curve are generated by serially diluting a stock sample used to perform a thermal gradient analysis. In some instances, the stock sample may be diluted, for example by 1:20, 1:10, 1:8, 1:4, or 1:2 prior to performing the serial dilutions. In some embodiments, a plurality of serial dilutions (typically between 5 to 10) is analyzed using, for example, qPCR and the quantification cycle (Cq) values plotted as a function of the log of the starting concentration (where lower Cq values correlate to higher target expression in a sample). In some cases, a linear regression analysis may be performed on the Cq versus log concentration plot to yield the reaction efficiency of the primer set. In some embodiments, the reaction efficiency is between 90% and 110%. For example, in some embodiments, the reaction efficiency of the primer set is greater than or equal to 90%, is greater than or equal to 95%, is greater than or equal to 100%, is greater than or equal to 105%, is greater than or equal to 110%, etc. In other embodiments, the reaction efficiency of the primer set is less than or equal to 110%, less than or equal to 105%, less than or equal to 100%, less than or equal to 95%, less than or equal to 90%, etc.

In some embodiments, the forward primer sequence comprises TCTGGACGGACGCTTCGGACGATGGAACAATTCAGTG (SEQ ID NO: 1). In some embodiments, the forward primer sequence comprises AGCGGTGTATACGGTGTAAACACTTCGACGCTTTCCGG (SEQ ID NO: 2). In some embodiments, the forward primer sequence comprises AGTGCGACATGGTACTTTTCTGTGATCGCTCGCCTCG (SEQ ID NO: 3). In some embodiments, the forward primer sequence comprises CCGCAAGCCGCTCCTTGAATCTACGGAGAGACTCAC (SEQ ID NO: 4). In some embodiments, the forward primer sequence comprises AATCGTCGCCGTCCTCACAAAAACAACCGCCG (SEQ ID NO: 5). In some embodiments, the forward primer sequence comprises CGCCGAGGCTAAATCGCAATCTACCTGACGTTCCTGTG (SEQ ID NO: 6). In some embodiments, the forward primer sequence comprises ATCGACTTGCCTGCTGTCATTACTTCACGCTCACTCCG (SEQ ID NO: 7). In some embodiments, the forward primer sequence comprises CGTACAGTGACCTATCGCCAGAATCTCACGCCAACAGC (SEQ ID NO: 8). In some embodiments, the forward primer sequence comprises CGGAGAAGCCAATCAGGTCCTTGATTCTCTACCAGCGC (SEQ ID NO: 9). In some embodiments, the reverse primer sequence comprises TTTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTTTTTTGCCGCCCACTCAGACTTTATTCAA AGACCACGGGG (SEQ ID NO: 10). In some embodiments, the reverse primer is used for amplification while simultaneously appending a poly-A tail onto the PCR product.

Aspects of the disclosure relate to methods for using an optimized primer set (e.g., a forward primer and a reverse primer) to amplify a DNA template fragment using, for example, a polymerase chain reaction (PCR). In some embodiments, the DNA template fragment may comprise a promoter region (e.g., T7 promoter), a 5′ UTR region, an ORF, a 3′ UTR region, and a poly-A tail region. In some embodiments, the amplified DNA template fragments may be subsequently assembled (e.g., stitched using Type IIS enzymes) into a target DNA strand, for example, to produce a mRNA vaccine or therapeutic. In some embodiments, the method comprises performing a plurality of polymerase chain reactions comprising a denaturation step (e.g., to separate the dsDNA template fragment into separate strands), an annealing step (e.g., to allow binding of forward and reverse primers to the separate DNA strands), and an elongation step (e.g., to allow DNA polymerase to assemble a daughter DNA strand).

In some cases, the methods comprise hybridizing a plurality of single stranded DNA template fragments (e.g., formed during denaturation step) with a first set of primers (e.g., a forward primer and a reverse primer) to produce a first set of DNA template-first primer duplexes (i.e., annealing step). The first set of primers can comprise a polynucleotide sequence having a guanosine or a cytidine at a 3′ terminal, and at least one nucleotide that is unique to that primer and different from each of the other primers in the first set of primers.

In some cases, the methods comprise extending a first set of DNA template-first primer duplexes in a 5′ to 3′ direction and a 3′ to 5′ direction to produce a first extension product (i.e., elongation step to produce the daughter strands). Accordingly, in some embodiments, the methods comprise providing a plurality of deoxynucleoside triphosphates (dNTPs) such as ATP, GTP, CTP, and UTP, and a DNA polymerase (e.g., Taq polymerase) to extend the first set of the DNA template-first primer duplex (i.e., to synthesize a daughter DNA strand). In some embodiments, the method comprises providing one or more buffer solutions optimized to the specific polymerase used. In some embodiments, the buffer system may comprise one or more monovalent (e.g., potassium, sodium, etc.) and/or bivalent cations (e.g., calcium, magnesium, manganese, etc.).

In some embodiments, the methods comprise hybridizing single strands (e.g., formed during the denaturation step) of the first extension product to a second set of primers, to produce a second set of DNA template-second primer duplexes (i.e., annealing step). The second set of primers, according to some embodiments, comprise a polynucleotide sequence having a guanosine or a cytidine at a 3′ terminal end, and at least one nucleotide that is unique to that primer and different from each of the other primers in the second set of primers.

In some cases, the methods comprise extending a second set of DNA template-second primer duplexes in both the 5′ to 3′ direction and the 3′ to 5′ direction to produce a second extension product (i.e., elongation step). In other cases, the methods comprise allowing the first extension product to hybridize with the second extension product in both the 5′ to 3′ direction and the 3′ to 5′ direction to produce a DNA template fragment.

In some embodiments, the process of denaturation, annealing and elongation constitute a single cycle. Multiple cycles may be used to amply the DNA target to the desired valve. In some embodiments, the number of DNA copies formed after “n” number of cycles may be calculated using the equation 2\hat{ }n. Thus, a reaction set for 30 cycles results in 2\hat{ }30 or 1,073,741,824 copies of the original double-stranded DNA template fragment. Therefore, in some embodiments, the number of cycles used to amplify the DNA template fragment is greater than or equal to 15 cycles, greater than or equal to 30 cycles, greater than or equal to 45 cycles, greater than or equal to 60 cycles, greater than or equal to 75 cycles, etc. In other embodiments, the number of cycles used to amplify the DNA template fragment is less than or equal to 75 cycles, less than or equal to 60 cycles, less than or equal to 45 cycles, less than or equal to 30 cycles, less than or equal to 15 cycles, etc.

### Nucleic Acids

Some aspects relate to compositions comprising nucleic acids and methods of producing nucleic acids. As used herein, the term “nucleic acid” includes multiple nucleotides (e.g., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, RNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

Nucleic acid sequences include nucleic acid sequences that have been removed from their naturally occurring environment and engineered nucleic acids. An “engineered nucleic acid” is a nucleic acid that does not occur in nature. It should be understood, however, that while an engineered nucleic acid as a whole is not naturally occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids, or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators) and is in a form suitable for use within genetically engineered protein production systems. Such isolated molecules are those that are separated from their natural environment.

In some embodiments, a nucleic acid is a DNA template for IVT. An “in vitro transcription template” (IVT template), or “DNA template” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the DNA template may be incorporated within a nucleic acid vector, which may be a circular nucleic acid such as a plasmid. In other embodiments it is a linearized DNA.

A DNA template may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes an RNA including at least the following elements: a 5′ untranslated region, an open reading frame region encoding the mRNA, a 3′ untranslated region and a polyA tail. The open reading frame may encode any mRNA sequence, or portion thereof. The DNA may be single-stranded or double-stranded. In some embodiments, the DNA is present on a plasmid or other vector. A DNA may include a polynucleotide encoding a polypeptide of interest. A DNA, in some embodiments, includes an RNA polymerase promoter (e.g., a T7 RNA polymerase promoter) located 5′ from and operably linked to a polynucleotide encoding a polypeptide of interest.

The length of the DNA, and thus the length of the RNA of interest which it encodes, may vary. For example, the DNA (and/or the RNA of interest) may have a length of about 200 nucleotides to about 10,000 nucleotides. In some embodiments, the DNA (and/or the RNA of interest) has a length of 200-500, 200-1000, 200-1500, 200-2000, 200-2500, 200-3000, 200-3500, 200-4000, 200-4500, 200-5000, 200-5500, 200-6000, 200-6500, 200-7000, 200-7500, 200-8000, 200-8500, 200-9000, or 200-9500 nucleotides. In some embodiments, the DNA (and/or the RNA of interest) has a length of at least 200, at least 300, at least 400, at least 500, at least 600, at least 700, at least 800, at least 900, at least 1000, at least 2000, at least 3000, at last 4000, at least 5000, at least 6000, at least 7000, at least 8000, at least 9000, or at least 10,000 nucleotides.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation comprising a plurality of copies of the molecule (e.g., DNA) of interest, for example a cell extract preparation comprising a plurality of expression vectors encoding a molecule of interest (e.g., a DNA encoding an RNA of interest).

A nucleic acid typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside. It should be understood that the term “nucleotide” includes naturally occurring nucleotides, synthetic nucleotides and modified nucleotides, unless indicated otherwise. Examples of naturally occurring nucleotides used for the production of RNA, e.g., in an IVT reaction, include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U), and 2′-O-methyl uridine. In some embodiments, an RNA transcript (e.g., mRNA transcript) includes a combination of at least two (e.g., 2, 3, 4 or more) of the foregoing modified nucleobases.

### In Vitro Transcription

The purified DNA template produced using the methods disclosed herein is of optimal high quality and is thus useful in the production of mRNA in an in vitro transcription (IVT) reaction. Aspects of the present disclosure provide methods of producing (e.g., synthesizing) an RNA transcript (e.g., mRNA transcript) comprising contacting a DNA template with an RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, the concentration of DNA in an IVT reaction mixture is about 0.01-0.10 mg/mL, 0.01-0.09 mg/mL, 0.01-0.075 mg/mL, 0.025-0.075 mg/mL, 0.01-0.05 mg/mL, 0.02-0.08 mg/mL, 0.02-0.06 mg/mL, 0.03-0.055 mg/mL, 0.04-0.05 mg/mL, or 0.05 mg/mL. In some embodiments, the concentration of DNA is maintained at a concentration of above 0.01 mg/mL during the entirety of an IVT reaction. In some embodiments, the concentration of DNA is maintained at a concentration is about 0.01-0.10 mg/mL, 0.01-0.09 mg/mL, 0.01-0.075 mg/mL, 0.025-0.075 mg/mL, 0.01-0.05 mg/mL, 0.02-0.08 mg/mL, 0.02-0.06 mg/mL, 0.03-0.055 mg/mL, or 0.04-0.05 mg/mL during the entirety of an IVT reaction.

In some embodiments, an IVT reaction uses an RNA polymerase selected from the group consisting of T7 RNA polymerase, T3 RNA polymerase, K11 RNA polymerase, and SP6 RNA polymerase. In some embodiments, an IVT reaction uses a T3 RNA polymerase. In some embodiments, an IVT reaction uses an SP6 RNA polymerase. In some embodiments, an IVT reaction uses a K11 RNA polymerase. In some embodiments, an IVT reaction uses a T7 RNA polymerase. In some embodiments, a wild-type T7 polymerase is used in an IVT reaction. In some embodiments, a mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference.

T7 RNA polymerase variants with one or more mutations relative to WT T7 RNA polymerase have several advantages in IVT reactions, including improved speed, fidelity, and reduced production of double-stranded RNA (dsRNA) transcripts. Double-stranded RNA transcripts, in which at least a portion of an RNA transcript is hybridized to another RNA molecule, elicit an innate immune response when introduced into a cell. Minimizing the formation of dsRNA transcripts during IVT enables the production of less immunogenic RNA compositions.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes an RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) region located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

Some embodiments comprise performing a co-IVT reaction that includes multiple input DNAs (or populations of input DNAs). In some embodiments, each input DNA (e.g., population of input DNA molecules) in a co-IVT reaction is obtained from a different source (e.g., synthesized separately).

An RNA transcript, in some embodiments, is the product of an IVT reaction. An RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide. In some embodiments, an RNA transcript produced by IVT is further modified by circularization, in which two non-adjacent nucleotides (e.g., 5′ and 3′ terminal nucleotides) of a linear RNA are ligated to produce a circular RNA with no terminal nucleotides.

The nucleoside triphosphates (NTPs) may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1.

The same IVT reaction may include 3.75 millimolar cap analog (e.g., trinucleotide cap or tetranucleotide cap). In some embodiments, the molar ratio of the cap to any of G, C, U, or A is 1:1. In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a IVT reaction is calculated empirically. For example, the rate of consumption for each NTP in an IVT reaction may be empirically determined for each individual input DNA, and then balanced ratios of NTPs based on those individual NTP consumption rates may be added to a IVT comprising multiple of the input DNAs.

In some embodiments, the IVT reaction mixture comprises one or more modified nucleoside triphosphates. In some embodiments, the IVT reaction mixture comprises one or more modified nucleoside triphosphates selected from the group consisting of N6-methyladenosine triphosphate, pseudouridine (v) triphosphate, 1-methylpseudouridine (m¹ψ) triphosphate, 5-methoxyuridine (mo⁵U) triphosphate, 5-methylcytidine (m⁵C) triphosphate, α-thio-guanosine triphosphate, and α-thio-adenosine triphosphate. In some embodiments, the IVT reaction mixture comprises N6-methyladenosine triphosphate. In some embodiments, the IVT reaction mixture comprises pseudouridine triphosphate. In some embodiments, the IVT reaction mixture comprises 1-methylpseudouridine triphosphate. In some embodiments, the concentration of modified nucleoside triphosphates in the reaction mixture is about 0.1% to about 100%, about 0.5% to about 75%, about 1% to about 50%, or about 2% to about 25%. In some embodiments, the concentration of modified nucleoside triphosphates is about 1%, about 2%, about 3%, about 4%, about 5%, about 6%, about 7%, about 8%, about 9%, about 10%, about 15%, about 20%, or about 25%.

In some embodiments, an RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine, and α-thio-adenosine. In some embodiments, an RNA transcript (e.g., mRNA transcript) includes a combination of at least two (e.g., 2, 3, 4 or more) of the foregoing modified nucleobases.

In some embodiments, an RNA transcript (e.g., mRNA transcript) includes pseudouridine (ψ). In some embodiments, an RNA transcript (e.g., mRNA transcript) includes 1-methylpseudouridine (m¹ψ). In some embodiments, an RNA transcript (e.g., mRNA transcript) includes 5-methoxyuridine (mo⁵U). In some embodiments, an RNA transcript (e.g., mRNA transcript) includes 5-methylcytidine (m⁵C). In some embodiments, an RNA transcript (e.g., mRNA transcript) includes α-thio-guanosine. In some embodiments, an RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention. In some embodiments, modified nucleotides are included in an IVT mixture, and are incorporated randomly during transcription, such that the RNA contains a mixture of modified nucleotides and unmodified nucleotides.

The buffer system of an IVT reaction mixture may vary. In some embodiments, the buffer system contains Tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of Tris is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids of the present disclosure as flanking regions to an ORF. This publication is incorporated by reference herein for this purpose.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8 (3): 568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo (dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids. Incorporation of intronic sequences can increase protein production as well as nucleic acid expression levels. In some embodiments, the nucleic acid comprises an internal ribosome entry site (IRES) instead of or in addition to a UTR (see, e.g., Yakubov et al., Biochem. Biophys. Res. Commun. 2010 394 (1): 189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In some non-limiting examples, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

Some aspects relate to methods of producing RNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The polyA-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail. The efficiency (e.g., percentage of polyA tail-containing RNAs in an RNA composition may be measured i) after the IVT reaction and before purification, or ii) after the RNA composition has been purified (e.g., by chromatography, such as oligo-dT chromatography).

Unique polyA tail lengths can provide certain advantages to the nucleic acids. Generally, the length of a polyA tail, when present, is greater than 30 nucleotides in length. In ssome embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA-binding protein can enhance expression.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A nucleic acid primer, comprising: a nucleic acid having a 5′ terminal end and a 3′ terminal end and a polynucleotide sequence of 20 to 40 nucleotides, wherein the polynucleotide sequence comprises a guanosine or a cytidine at the 3′ terminal end.

2. The nucleic acid primer of claim 1, wherein the polynucleotide sequence has between about 32 and 38 nucleotides.

3. The nucleic acid primer of any one of claims 1-2, wherein the polynucleotide sequence comprises a GC content of about 50%.

4. The nucleic acid primer of any one of claims 1-3, wherein the polynucleotide sequence has a low primer-homodimer complex forming propensity.

5. The nucleic acid primer of claim 4, wherein the primer-homodimer complex forming propensity comprises a AG of greater than or equal to −3.0 kcal/mol.

6. The nucleic acid primer of any one of claims 1-5, wherein the polynucleotide sequence has a low hairpin structure forming propensity.

7. The nucleic acid primer of claim 6, wherein the hairpin structure forming propensity comprises a AG of greater than or equal to −2.5 kcal/mol.

8. The nucleic acid primer of claim 1, wherein the polynucleotide sequence is
(SEQ ID NO 1)
TCTGGACGGACGCTTCGGACGATGGAACAATTCAGTG.

9. The nucleic acid primer of claim 1, wherein the polynucleotide sequence is
(SEQ ID NO 2)
AGCGGTGTATACGGTGTAAACACTTCGACGCTTTCCGG.

10. The nucleic acid primer of claim 1, wherein the polynucleotide sequence is
(SEQ ID NO 3)
AGTGCGACATGGTACTTTTCTGTGATCGCTCGCCTCG.

11. The nucleic acid primer of claim 1, wherein the polynucleotide sequence is
(SEQ ID NO 4)
CCGCAAGCCGCTCCTTGAATCTACGGAGAGACTCAC.

12. The nucleic acid primer of claim 1, wherein the polynucleotide sequence is
(SEQ ID NO 5)
AATCGTCGCCGTCCTCACAAAAACAACCGCCG.

13. The nucleic acid primer of claim 1, wherein the polynucleotide sequence is
(SEQ ID NO 6)
CGCCGAGGCTAAATCGCAATCTACCTGACGTTCCTGTG.

14. The nucleic acid primer of claim 1, wherein the polynucleotide sequence is
(SEQ ID NO 7)
ATCGACTTGCCTGCTGTCATTACTTCACGCTCACTCCG.

15. The nucleic acid primer of claim 1, wherein the polynucleotide sequence is
(SEQ ID NO 8)
CGTACAGTGACCTATCGCCAGAATCTCACGCCAACAGC.

16. The nucleic acid primer of claim 1, wherein the polynucleotide sequence is
(SEQ ID NO 9)
CGGAGAAGCCAATCAGGTCCTTGATTCTCTACCAGCGC.

17. A method of preparing a DNA template fragment, comprising:
(i) hybridizing a plurality of DNA templates with a first set of primers to produce a first set of DNA template-first primer duplexes, wherein the first set of primers comprises two or more primers, and wherein each of the primers in the first set of primers comprises a polynucleotide sequence having a guanosine or a cytidine at a 3′ terminal end, and at least one nucleotide that is unique to that primer and different from each of the other primers in the first set of primers;
(ii) extending the first set of DNA template-first primer duplexes in a 5′ to 3′ direction and a 3′ to 5′ direction to produce a first extension product;
(iii) hybridizing single strands of the first extension product to a second set of primers, to produce a second set of DNA template-second primer duplexes, wherein the second set of primers comprises two or more primers, and wherein each of the primers in the second set of primers comprises a polynucleotide sequence having a guanosine or a cytidine at a 3′ terminal end, and at least one nucleotide that is unique to that primer and different from each of the other primers in the second set of primers;
(iv) extending the second set of DNA template-second primer duplexes in both the 5′ to 3 direction and the 3′ to 5′ direction to produce a second extension product; and
(v) allowing the first extension products to hybridize with the second extension products in both the 5′ to 3′ direction and the 3′ to 5′ direction to produce the DNA template fragments.


SEQUENCE LISTING (10 sequences)

SEQ ID NO: 1 - DNA, 37 bp, synthetic construct
  source 1..37: mol_type = other DNA
tctggacggacgcttcggacgatggaacaattcagtg

SEQ ID NO: 2 - DNA, 38 bp, synthetic construct
  source 1..38: mol_type = other DNA
agcggtgtatacggtgtaaacacttcgacgctttccgg

SEQ ID NO: 3 - DNA, 37 bp, synthetic construct
  source 1..37: mol_type = other DNA
agtgcgacatggtacttttctgtgatcgctcgcctcg

SEQ ID NO: 4 - DNA, 36 bp, synthetic construct
  source 1..36: mol_type = other DNA
ccgcaagccgctccttgaatctacggagagactcac

SEQ ID NO: 5 - DNA, 32 bp, synthetic construct
  source 1..32: mol_type = other DNA
aatcgtcgccgtcctcacaaaaacaaccgccg

SEQ ID NO: 6 - DNA, 38 bp, synthetic construct
  source 1..38: mol_type = other DNA
cgccgaggctaaatcgcaatctacctgacgttcctgtg

SEQ ID NO: 7 - DNA, 38 bp, synthetic construct
  source 1..38: mol_type = other DNA
atcgacttgcctgctgtcattacttcacgctcactccg

SEQ ID NO: 8 - DNA, 38 bp, synthetic construct
  source 1..38: mol_type = other DNA
cgtacagtgacctatcgccagaatctcacgccaacagc

SEQ ID NO: 9 - DNA, 38 bp, synthetic construct
  source 1..38: mol_type = other DNA
cggagaagccaatcaggtccttgattctctaccagcgc

SEQ ID NO: 10 - DNA, 135 bp, synthetic construct
  source 1..135: mol_type = other DNA
ttttttttttttttttttttttttttttttttttttttttttttttttttttttttttttttttttttttttttttttttttttttttttttttttttttgccgcccactcagactttattcaaagaccacgggg